(19) United States
(12) Patent Application Publication
(10) Pub. No.: US 2021/0280304 A1
(43) Pub. Date: Sep. 9, 2021

(54) INTEGRATED CLINICAL TRIAL DESIGN AND DESIGN OPTIMIZATION

(71) Applicant: Gen LI, East Lyme, CT (US)

(72) Inventor: Gen LI, East Lyme, CT (US)

(21) Appl. No.: 17/194,434

(22) Filed: Mar. 8, 2021

Related U.S. Application Data
(60) Provisional application No. 62/986,746, filed on Mar. 8, 2020.

(51) Int. Cl. G16H 40/20; G16H 10/20; G16H 70/60; G16H 10/60; G16H 50/70; G06F 16/25

(52) U.S. Cl. CPC G16H 40/20; G16H 10/20; G16H 70/60; G06Q 10/06375; G16H 50/70; G06F 16/258; G16H 10/60

(57) ABSTRACT

Clinical trial protocols frequently need to be amended during a clinical trial, but the amendments are costly and can delay new drug approval. It is challenging to avoid, reduce or minimize incidences of amendments. Currently, people have to manually identify flaws of protocol, which is slow and may incur errors due to subjective perspectives and lack of objective, sufficient, and structured data. The present invention discloses an integrated clinical trial design and design optimization platform that efficiently reduces/minimizes incidences of amendments. In one embodiment, the protocol design or optimization is based on data. In one embodiment, the result is more accurate, quantitative and less error-prone; the system can quickly identify flaws; the flaws can be detected at an earlier stage and the system can distinguish key and leading factors and variables, helping sponsors identify the most efficient amendment.

### CROSS-REFERENCE TO RELATED APPLICATIONS

This application claims the benefit of U.S. Ser. No. 62/986,746, filed Mar. 8, 2020. Throughout this application, various references or publications are cited. Disclosures of these references or publications in their entireties are hereby incorporated by reference into the application in order to more fully describe the state of the art to which this invention pertains.

### FIELD OF THE INVENTION

This invention relates to systems and methods for integrated clinical trial design and design optimization.

### BACKGROUND OF THE INVENTION

Clinical trial protocols currently are designed by protocol writers, usually medical professionals, based on their personal, medical and other knowledge. The protocols are prone to various errors and mistakes and hence have to be updated and modified while being implemented. Such updates and modifications (also called protocol amendments) are common and happen frequently. Protocol amendments often happen multiple times in a single clinical trial. These amendments are very costly, about $300,000 each, and they slow down clinical trials, delaying lifesaving medicines to reach patients' bedside. Since clinical trial protocol amendments are costly if they happen frequently, it is highly challenging to reduce and/or minimize incidences of protocol amendments. This is one of the issues the present invention intends to overcome.

There are existing solutions, but they are largely manual, vary by writers and are error-prone. Those existing solutions require human involvement in manually identifying flaws of current protocol and amending protocols. This manual process is slow and may probably incur errors due to subjective perspectives and lack of objective, sufficient, and structured data. It is known that many clinical trials found on clinicaltrials.gov, the platform sanctioned by the USA government for publicizing clinical trials in various stages, as well as those from other regional and country clinical trial registries, are flawed and mandates protocol amendments. By utilizing the platform for integrated clinical trial design and design optimization as described in the present invention, the flaws of protocol design can be easily identified. Another advantage of the platform is its ability of detecting flaws at an early stage, usually before the patient recruitment stage for some trials, whereas existing methods for protocol amendments usually can only do so with at least some clinical trial results, for example, after the clinical trials begin.

Database is a commonly used tool for data analysis. However, it is unpractical to understand the whole set of data and their meaning, especially when the data comprises a large volume of data sometimes in different formats. It is neither practical nor efficient to do an analysis merely based on a set of data. In one aspect, the present invention provides a computer-based and visual platform listing all or substantially all key information for clinical trial protocol evaluation and amendment. Such visual platform demonstrates a number of advantages including better efficiency, faster speed, improved accuracy and less error. In another aspect, the platform as disclosed in the present invention provide a set of baseline characteristics based on filtered data from all associated historical clinical trials. In one embodiment, the present invention provides a graphical user interface-based platform for a quantitative analysis quantifying the potential effect(s) caused by an adjustment.

### SUMMARY OF THE INVENTION

The present invention of a platform for integrated clinical trial design and design optimization provides a data driven, communicable framework of methods and/or systems to speed up the clinical trial process with dramatically improved quality. It is proven to reduce and minimize the incidence of protocol amendments. It has also proven that this platform allows new medicine developers to objectively simplify clinical trial design, improve cost effectiveness in bringing life-saving new medicine to reach patients sooner.

Features of the present invention are as follows.
It is structured and thorough;
It is quantitative, objective and easily communicable among stakeholders, including regulatory agencies such as the US Food and Drug Administration (FDA), or other regulatory authorities around the world;
It is less dependent of protocol writers' personal experience, and thus less prone to various errors and mistakes; and
It enables more bold medical innovations, since it anchors a large number of variables and is more focused on the innovative variables.

Compared to existing solutions, the present invention has advantages as follows.
It is based on data, or data driven, so that it can avoid biases;
It is quicker to identify flaws in clinical trial protocols, since it is not manually operated;
It is more accurate, because it is quantitative, and less error-prone;
It can detect flaws at an earlier stage of a trial, for example, before the patient enrollment stage; and
It can reveal key leading factors and/or variables that help sponsors to focus on more efficient ways of improving and/or amending a trial protocol.

### BRIEF DESCRIPTION OF THE FIGURES

FIG. 1 is a bar chart of row count versus eligibility minimum age, illustrating the concept of modal value.

FIG. 2 is a bar chart of row count versus ECOG PS, illustrating the concept of modal value.

FIG. 3 is a plot of Gross Site Enrollment Rate (GSER, defined as patients enrolled per site per month) versus the number of investigator sites for a group of COPD (chronic obstructive pulmonary disease) clinical trials. The plot is a “bubble chart”, wherein each bubble or sphere represents a clinical trial, and the size of the bubble represents the enrollment cycle time.

### DETAILED DESCRIPTION OF THE INVENTION

The following terms shall be used to describe the present invention. In the absence of a specific definition set forth herein, the terms used to describe the present invention shall be given their common meaning as understood by those of ordinary skill in the art.

“Mode value”, or “modal value” is a concept from statistics. In a group of samples with various values, the most frequent value is called the modal value. For example, suppose in a Phase 2 Non-small Cell Lung Cancer (NSCLC) clinical trials, most of the trials include patients who are 18 years old or older, which is the most common age considered to be an adult. A few other trials from Japan may include patients who are 21 years old, where it is the legal adult age or older. Another few trials from Ireland may include patients who are 16 years old or older. In this example, the modal value for the minimum age is 18 years old.

Sometimes the regulatory agency may ask a sponsor to do a trial only in geriatric population, which is 65 years old or older. Despite the fact that the majority of NSCLC patients are 65 or older, the trial will become more difficult beyond the linear exclusion of those who are under 65 years old. In the past, the trial sponsor could only complain orally about this negative impact on the trial, but FDA usually ignored such complaints. The present invention of a platform for integrated clinical trial design and design optimization will allow the sponsor to present a quantitative view of that impact to any stakeholders, including the FDA.

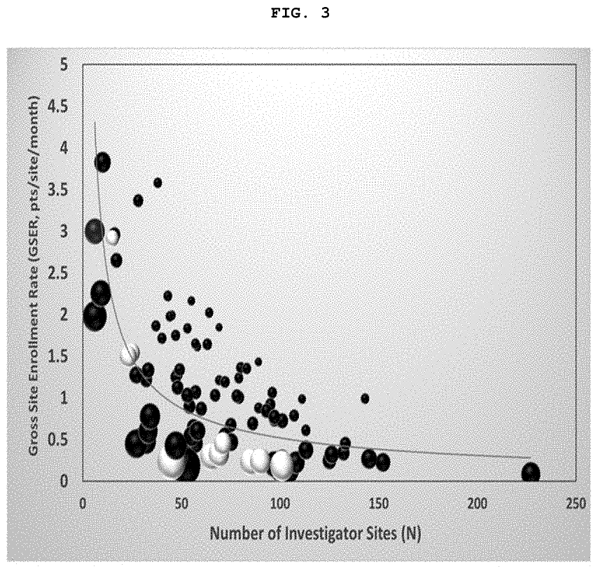

Forced Expiratory Volume (FEV1) is the volume of air that can be forcibly blown out in one second. FEV1 value is an important indicator when evaluating chronic obstructive pulmonary disease (COPD) and monitoring progression of the condition. FEV1 among COPD patients, as a percentage of normal level, is commonly from 30% to 80%, which is the modal value in COPD clinical trials according to the inventor's analysis on selected data. If a COPD trial is now targeting to enroll COPD patients with FEV1 from 30% to 65% of normal level, it would result in a narrower patient population, and the enrolled patients are much sicker on average. As shown by the white (clear) bubbles in FIG. 3, the clinical trial becomes much more difficult to conduct, because it is expected to have a lower enrollment rate (typically measured by GSER), the GSER value is expected to decrease from 0.99 patient per site per month to 0.55 patient per size per month, and the enrollment cycle time corresponding to larger sizes of bubbles is expected to increase from 306 days to 678 days. It indicates that a protocol amendment would be needed for the FEV1 value, and its value shall be adjusted to the modal value(s) based on the above analysis.

A “clinical trial protocol”, or simply a “trial protocol” or a “protocol”, comprises a set of parameters and operational deliverables used to guide the setup and/or the implementation of a clinical trial. Designing a protocol is a process of putting the protocol together by setting those parameters and [expected] deliverables, and optimizing a protocol is the process of fining the optimal set of values for those parameters and deliverables so that the clinical trial can be conducted most efficiently and conclusively. The optimization is conducted in light of the objective and priority of a clinical trial.

To the best knowledge of the inventor, there is no existing system for protocol design or optimization. The present invention for the first time provides a system capable of designing a protocol, identifying potential flaws in the protocol for protocol amendments, and/or optimizing the protocol by reducing/minimizing the incidence of protocol amendments.

Operational parameters and operational deliverables may deviate from the modal values, and this can have a real and material impact on the protocol design. One of the ways to measure this impact is by the number of amendments needed when a trial is carried out. The present invention can measure the impact or alike without waiting for a long time or for the trial to be substantively carried out.

A “clinical trial parameter”, or a “protocol parameter”, is a parameter involved in the setup and/or implementation of a clinical trial. Examples of clinical trial parameters include a subject's age, Body Mass Index (BMI), etc.

“Enrollment rate” measures how fast and how efficiently a site or clinical trial enrolls patients within a unit amount of time (e.g., a month or a season).

“Enrollment Cycle Time” is the period starting from enrollment opening date and ended with enrollment closing date, (Etₛ−Stₛ). In one embodiment, ECT can be calculated as:
Enrollment Cycle Time=Total Enrollment/[(Gross Site Enrollment Rate(GSER)×(Maximum Number of Investigator Sites(Nₘₐₓ)],
   wherein the GSER is related to site selection (performance), among other things, and SEI is related to study startup (process).

In one embodiment, GSER means Gross Site Enrollment Rate and is defined as number of patients enrolled per site per unit of time. In one embodiment, CTER means Clinical Trial Enrollment Rate and is defined as number of patients enrolled per unit of time.

“Site Effectiveness Index” is defined as:

$$SEI = \frac{{\int }_{i = 1}^{N} ( {Et}_{i} - {St}_{i} )}{( {Et}_{s} - {St}_{s} ) \times {N}_{max}} ,$$

wherein Etᵢ is the time (date) site i closed for patient enrollment, Stᵢ is the time (date) site i opened for patient enrollment, Nₘₐₓ is the maximum number of investigator sites opened for enrollment during the patient enrollment of the study (trial), Etₛ is the time (date) clinical study (trial) closed for patient enrollment, Stₛ is the time (date) clinical study (trial) opened for patient enrollment. Etₛ is the time (date) clinical study (trial) ended for patient enrollment.

In one embodiment, “Adjusted Site Enrollment Rate” (ASER) is defined as:

$$ASER = \frac{TE}{{\int }_{i = 1}^{N} ( E {t}_{i} - S {t}_{i} )}$$

wherein TE is Total Enrollment. When the target clinical trial is in the planning stage, TE refers to the targeted total number of patients to be enrolled in the target clinical trial. For an evaluation of historical data, TE is the total number of patients actually enrolled in a clinical trial.

In one embodiment, “Gross Site Enrollment Rate” (GSER) is related to Site Effectiveness Index (SEI) and Adjusted Site Enrollment Rate (ASER) as: GSER=SEI×ASER.

A “synthesized parameter” is a parameter derived from a set of other variables or parameter values. For example, GSER is a synthesized parameter derived from number of enrolled patients, number of sites and enrollment cycle time and can be calculated as number of patients enrolled per site per unit of time.

“Baseline patient statistics” is a summary of the characteristics of recruited eligible patients at the beginning of the trial.

A “subject”, or a “patient”, refers to an individual enrolled into a clinical trial at an investigator site.

A “master database” is a database that stores data related to clinical trials, subjects/patients, and other related information for all disease/syndromes and indications.

A “user-defined filter”, or simply a “user filter” or a “filter”, is a selection rule that generates a subset of selected data from the master database and such selected data is optionally stored in a sub-database.

A “relational database” is a digital database based on the relational model of data.

“Integrated structure” in the present invention refers to the organization, analysis and use of clinical trial data and the relationships among [important] variables such that a stakeholder may easily use same to evaluate clinical trial protocol and/or amendment in an efficient way. Making such evaluation is typically impossible if the data is not organized, analyzed, or presented by the integrated structure of the present invention.

Actual steps can be taken to amend the protocol to allow the clinical trial to get back on the right track. It would be even more efficient to take the same steps before the implementation so the amendments can be avoided.

In one embodiment, the present invention provides a method for identifying flaws in a clinical trial protocol, the method comprising the steps of:
(a) creating a selected data or sub-database by applying a user-defined filter to a master database, wherein the filter comprises a set of filtering parameters, and the sub-database contains clinical trials related to the disease or condition, and has sufficient data for subsequent analysis;
(b) from the selected data which is optionally stored in a sub-database, identifying each and every parameter, such parameters include but are not limited to gender, age (minimum and/or maximum age), race, type/stage of disease/disorder, phase of the clinical trial, country, number of patients, number of investigator sites, ECT, SEI, GSER, CTER, ASER and any other pre-set parameters fitting objectives and features of a clinical trial in view of the objectives of the target clinical trial and/or particular needs such as sufficiency of data for analysis;
(c) mapping out relationship between each parameter and final outcome;
(d) for a target clinical trial, evaluating impact of certain parameter (either single parameter, a set of multiple parameters, or a synthesized parameter representing multiple parameters) on the final outcome:
   (1) if certain parameter would likely to lead to unfavorable/unacceptable impact (e.g., side effect) or any other result that fails the clinical trial, identify the parameter and/or its value as a flaw;
   (2) if certain parameter would be suitable for such clinical trial, identify it as a parameter not subject to amendment;
(e) among those identified flaws, conduct quantitative analysis and further identify the top important parameters subject to amendment; and
(f) adjusting/amending clinical trial parameters for the target clinical trial accordingly.

In one embodiment, the relationship between a (synthesized) parameter and the outcome can be described as a mathematical formula. In one embodiment, the relationship can be expressed as a chart. In one embodiment, the outcome can include but not limited to the patient enrollment efficiency (for example, as measured by GSER and ASER), the patient enrollment time, the budget for patient enrollment, the budget for patient enrollment and clinical trial, and any other objectives of the target clinical trial.

In one embodiment, patient data can be non-discriminately collected around the world regardless of disease conditions, geographic coverage, timeline, type of sponsors and etc. In one embodiment, data collection may be focused on more recent data.

In one embodiment, the selected data or sub-database can be generated by applying query according to a predefined set of standards to align best with the protocol/clinical trial being planned or evaluated.

In one embodiment, the present invention provides a system for identifying flaws in a clinical trial protocol for amendment, the system comprising:
1) A user-defined filter applied to a master database, wherein said filter comprises a set of filtering parameters used to create selected data which is optionally saved in a sub-database, wherein such selected data is related to the disease or condition, and contains sufficient data for subsequent analysis;
2) An identifying unit that identifies the existing variables for inclusion and exclusion criteria in the selected data;
3) An engine that explores the relationship between variables [or synthesized parameters] and the outcome of the clinical trials, wherein such engine stores and/or tabulates the relationships into a relational database;
4) An evaluation unit, wherein such evaluation unit, in view of the relationships, evaluates whether the existence or the value of variable in a protocol of a target clinical trial is a flaw and exports a proposed amendment for amending the protocol so as to improve the efficiency of the target clinical trial.

In one embodiment, the master database and the selected data (sub-database) are stored separately. In one embodiment, the relationships can be only revealed to certain client without giving access to others so that the client's information including these relationships and filtering information will not be subject to any unintentional disclosure risks. In one embodiment, the selected data or the sub-database is encrypted. In one embodiment, the master database is also encrypted. In one embodiment, different encryption strategies or approaches are adopted so that the access is user-specific.

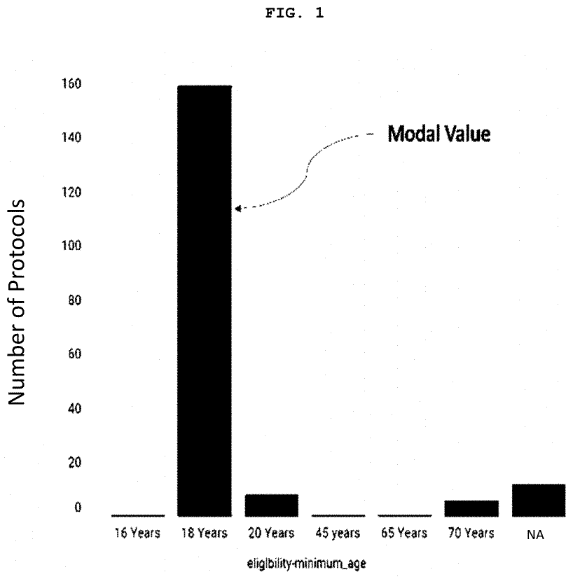

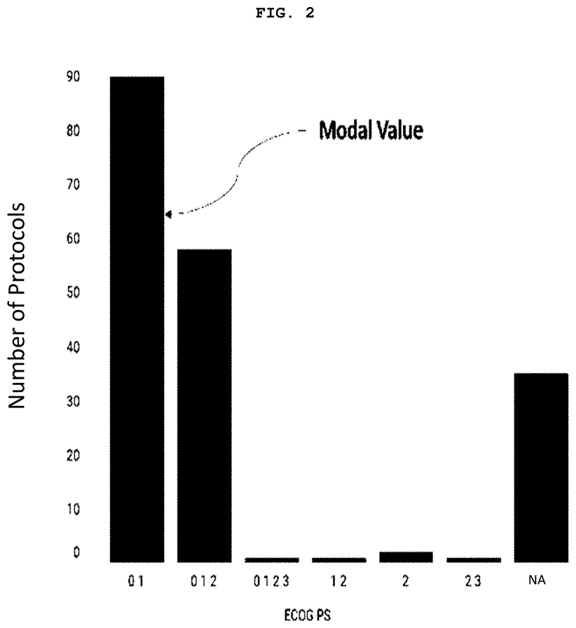

In one embodiment, FIG. 1 and FIG. 2 representatively show how to determine the modal value.

In one embodiment, FIG. 3 typically shows an established relationship between the number of investigator sites (N) and the GSER which can be used to evaluate the potential impact of N as a parameter on the patient enrollment efficiency, i.e., GSER as an outcome/deliverable of such clinical trial.

Sensitivity analysis is an analysis conducted for mapping out the relationship between parameter and outcome, and/or identifying the importance of a variable adjustment to the outcome. In one embodiment, the sensitivity analysis is driven by protocol amendment data in related clinical trials. In one embodiment, the sensitivity analysis is conducted by comparing the influence of different values for one variable so as to find the optimal value for such variable. In one embodiment, the sensitivity analysis is conducted by comparing the influence of different parameters with their optimal values so as to locate the more important variable(s).

In one embodiment, the validation means is driven by synthesized baseline patient characteristics.

In one embodiment, a clinical trial protocol is perfected by the platform for integrated clinical trial design and design optimization before it is conducted.

In one embodiment, the original set of parameter values as those included in the protocol eventually, one step of a time, converge to the “modal values”, but they do so at the expense of 4 different protocol amendments due to the flaws in the trial protocol, costing $1.2 million and significant delays in the trial. With the present invention of a platform for integrated clinical trial design and design optimization, these flaws would have been easily avoided at the beginning of the trial (e.g., before patient enrollment) so that both money and time would be saved.

In one embodiment, a database of clinical trials contains over 300,000 clinical trials, wherein only a portion of all the clinical trials is eventually successful.

In one embodiment, data of the subjects enrolled in clinical trials are obtained, analyzed, and synthesized onto the platform for integrated clinical trial design and design optimization.

Amending an ongoing clinical trial is time consuming and financially expensive. It is usually not implemented unless it is absolutely needed. This indicates that an accurate protocol design is most valuable for a clinical trial, whereas existing methods for clinical trial protocol design have this vulnerability of not rendering the protocol design accurate enough to minimize subsequent protocol amendments and thus is most vulnerable in successfully conducting a clinical trial. The present invention utilizes clinical trial data and other types of data accumulated over time to carefully fix the potential flaws proactively at all stages, especially early stages, of the clinical trials so as to avoid the existing methods' vulnerability.

Every successful clinical trial enrolls a group of patients from whom data can be gathered to illustrate the effects of the experimental intervention to this group of patients in terms of both the efficacy and safety. However, data usually can only be gathered after an expensive clinical trial is implemented. The present invention of a platform will allow its users to “synthesize” a group of patients at an early stage, for example, before the patient enrollment or implementation of any trial, which allows the users to validate all the elements/parameters of the protocol, such as patients' age, Body Mass Index (BMI), etc.

After completion of a clinical trial, sponsors are usually required to report the results, regardless of whether the trial has achieved its planned objectives or not. A report of the results from a clinical trial usually includes three key components as follows.
“Baseline patient characteristics”, which are defined as a summary of the characteristics of recruited patients at the beginning of the trial;
A summary of the impact of the experimental intervention in terms of its treatment efficacy. For example, the extent to which an antihypertensive agent lowers the blood pressure of a group of patients taking the agent; and
A summary of the impact of the experimental intervention in terms of patient safety, or any side effects. For the same example as above, other than lowering the blood pressure, the antihypertensive agents may cause dizzy, vomiting, and other side effects.

The regulatory approval of an experimental medicine is usually based on a balance between its efficacy and safety, as well as on its “superiority” over competing interventions already approved in the market.

In one embodiment, a database is built, tabulating the resulting patient characteristics data from those above-mentioned implemented clinical trials from various sources such as conference abstracts, oral presentations, publications, regulatory filings, etc., composed of over 14 million patients and over 30,000 clinical trial arms. The volume of the data is still rapidly growing. These data include a huge volume of information related to all the diseases for different populations. The present invention provides a new approach to, without conducting an actual clinical trial and extracting relevant information from the historical data, “synthesizing” the baseline patient characteristics for a population targeted by a clinical trial according to the need or priority being addressed by the clinical trial.

In one embodiment, the present invention can synthesize the baseline characteristics based on selected and sufficient data relating to the target population, such as sliced and/or diced data. From those slices and dices, an analysis can be conducted to explore the potential impact, therefore validating and perfecting the design of the protocol without actually implementing the clinical trial. In one embodiment, the present invention provides an approach of reducing/minimizing the incidence of protocol amendments. The clinical trial sponsor and the patients would benefit by the improved clinical trial and the approval of innovative medicine or treatment.

Currently in clinical development organizations around the world, problems are solved in isolation, and people carry out their work by at large in silo. For example, when one or more of investigator sites are not performing well, people try to find ways to improve the sites' performance. When a protocol requires a lot of amendments, it is probably due to the poorly designed protocol, and people have to try to find ways to improve the protocol design. With the platform in the present invention, variables are observed in an integrated structure. For example, poor site performance can be caused by a poor design that unnecessarily restricts certain eligible patients. When the enrollment rate is low and enrollment cycle time is long, the present invention can detect the deeply-rooted reasons related to the clinical trial processes, such as site activation, site performance, and/or protocol design. This integrated platform and approach allow the users to identify the true causes of the problems, therefore the present invention identifies and implements a fundamental solution.

An analogy is provided to illustrate the integrated structure of the platform in the present invention. Suppose 6,000 Lego pieces can be assembled into a model of Taj Mahal. Prior solutions to overcoming the issues with clinical trial design are similar to that, with all the 6,000 pieces scattered around and unorganized, it is practically impossible to find the two pieces corresponding to the top two corners of the front door of the Taj Mahal model. In this analogy, with an established relationship obtained from the analysis based on selected historical data, the method/platform as disclosed in the present invention can more easily, efficiently and accurately locate the targeted two pieces, even if the Taj Mahal model is not actually assembled or at an early stage of assembly. In one embodiment, the present invention provides a relational database mapping out the influence of input on output. With the relationship database, the present invention provides a quick and correct solution so as to achieve one or more objectives of the clinical trial. The objectives may include without limitation a specific population, enrollment time, a certain number of patients, a certain number of investigator sites, a confidence level, or a balance thereof.

In one embodiment, the present invention can generate a filter for seamless analysis of selected data so as to explore relationships between each protocol parameter and the outcome, wherein the selected data is generated by applying the filter to the general data or database. In one embodiment, the relationships as explored are stored as a relational database, which is operable with a computing unit for impact analysis and/or quantification. In one embodiment, the relational database comprises one or more tables and/or graphs showing the relationship.

In one embodiment, the filter is adjusted in view of the target clinical trial's objective and priority so as to collect selected data for subsequent analysis. In one embodiment, the filter is adjusted so as to investigate the influence of a filter parameter adjustment to the parameter-outcome relationships.

In one embodiment, the relationships are evaluated in view of the importance to the outcome or objective. In one embodiment, the protocol amendment shall be conducted by adjusting the top important parameters.

In one embodiment, the evaluation is conducted concurrently with or prior to the patient enrollment. In one embodiment, the evaluation is conducted concurrently with or during the patient enrollment or implementation of clinical trial.

In one embodiment, the evaluation can improve program efficiency across all phases of development and all therapeutic areas by 25-40%.

In one embodiment, the present invention can evaluate the indication of competition, success rates of alternate trial designs (e.g. adaptive vs. traditional), KOLs and high performing trialists.

In one embodiment, the present invention provides a method for evaluating and amending a protocol of a target clinical trial associated with a disease wherein said protocol has a plurality of parameters each having a value, comprising:
1) obtaining a set of inclusion and exclusion criteria of a target clinical trial associated with a disease;
2) transforming said criteria to a filter for filtering a master database storing patient data in historical clinical trials associated with said disease, generating a subdatabase storing filtered data;
3) constructing a set of baseline characteristics based on said filtered data stored in said subdatabase;
4) conducting a quantitative analysis by comparing said protocol to said set of baseline characteristics; and
5) making a recommendation based on said quantitative analysis; wherein said recommendation comprises one of the following:
   a) no protocol amendment is needed if said quantitative analysis indicates that said protocol would achieve one or more objectives of said target clinical trial; and
   b) a protocol amendment is needed if said quantitative analysis indicates one of the following:
      (1) at least one parameter of said protocol shall be removed or added; and
      (2) at least one parameter value of said protocol shall be adjusted,
   wherein said protocol amendment would help said target clinical trial achieve one or more objectives according to said quantitative analysis.

In one embodiment, the set of baseline characteristics comprises all parameters for patient screening in said historical clinical trials.

In one embodiment, the set of baseline characteristics comprises all key parameters with commonly used values for patient screening in said historical clinical trials.

In one embodiment, the commonly used values comprise modal values.

In one embodiment, the quantitative analysis quantifies one or more effects to said target clinical trial caused by an adjustment assuming said adjustment is made.

In one embodiment, the one or more effects are or associated with one or more of the following:
a) patient enrollment time,
b) number of patients,
c) number of investigator sites,
d) efficiency of enrolling patient at either site or clinical trial level,
e) cost, and
f) a confidence level to achieve any one of a)-e).

In one embodiment, the efficiency of enrolling patients at site level is characterized by Gross Site Enrollment Rate (GSER), wherein GSER is defined as number of patients enrolled per site per unit of time. In one embodiment, the efficiency of enrolling patients at site level is characterized by Adjusted Site Enrollment Rate (ASER), wherein ASER is defined as:

$$A S E R = \frac{T E}{{\int }_{i = 1}^{N} ( E {t}_{i} - S {t}_{i} )}$$

wherein TE is Total Enrollment, Etᵢ is the time or date the ith site was closed for patient enrollment, and Stᵢ is the time or date the ith site was opened for patient enrollment. In one embodiment, the efficiency of enrolling patients at site level is characterized by Site Effectiveness Index (SEI), wherein SEI is defined as:

$$SEI = \frac{{\int }_{i = 1}^{N} ( {Et}_{i} - {St}_{i} )}{( {Et}_{s} - {St}_{s} ) \times {N}_{max}} ,$$

wherein Etᵢ is the time or date the ith site was closed for patient enrollment, and Stᵢ is the time or date the ith site was opened for patient enrollment, Nₘₐₓ is the maximum number of investigator sites opened for enrollment during the patient enrollment, Etₛ is the time or date the historical clinical trial was closed for patient enrollment, Stₛ is the time or date the historical clinical trial was opened for patient enrollment.

In one embodiment, the efficiency of enrolling patients at clinical trial level is characterized by Clinical Trial Enrollment Rate (CTER), wherein CTER is defined as number of patients enrolled per unit of time.

In one embodiment, the set of baseline characteristics is provided in a table format.

In one embodiment, the set of baseline characteristics is constructed on a graphical user interface, which allows a user to, in view of importance and priority, add or remove one or more parameters being displayed on said graphical user interface.

In one embodiment, the graphical user interface virtually displays one or more quantitative analysis results upon selection by a user, wherein said one or more quantitative results are from said quantitative analysis.

In one embodiment, the quantitative result displayed on said graphical user interface comprises a bar chart, a bubble chart or both.

In one embodiment, the graphical user interface displays a bar chart and a bubble chart, wherein said bar chart profiles values of a selected parameter and frequencies of said values in historical trials, and wherein said bubble chart comprises a plurality of bubbles each representing one clinical trial, and size of bubble reflects enrollment cycle time, wherein when said user selects a value from said values for said selected parameter in said bar chart, the bubble chart highlights all clinical trials with said selected value for said selected parameter.

In one embodiment, the master database further stores other data from other sources comprising conference abstracts, oral presentations, publications, and regulatory filings.

In one embodiment, the other data from said other sources is tabulated and stored in a uniform format for data filtering.

In one embodiment, the one or more objectives comprise one or more of the following:
a) enrollment of patients is completed within a target time;
b) a target number of patients is enrolled,
c) a target number of investigator sites is registered,
d) a target efficiency of enrolling patients at either site or clinical trial level is achieved, e) a target budget is contained, and
f) a target confidence level to achieve any one of a)-e) is satisfied.

In one embodiment, when a protocol amendment is needed, the clinical trial protocol is amended by adjusting a parameter value to modal value in said set of baseline characteristics.

In one embodiment, when a protocol amendment is needed, said protocol is amended to achieve a new objective.

In one embodiment, the new objective is set in view of importance or priority of said target clinical trial from time to time.

In one embodiment, when a protocol amendment is needed, said protocol is amended in a way that minimum incidences of amendments would be needed.

In one embodiment, the present invention can evaluate indication feasibility, protocol feasibility, site feasibility, site selection, and implementation management.

In one embodiment, the present invention can evaluate comparable protocols, including but without limitation inclusion/exclusion criteria (including amendments) and impact on recruitment, high-performing countries, optimal number of sites and patients/site, scenarios for recruitment curve, and/or optimal site activation curve. In one embodiment, the present invention can optimize, or propose potential amendment for a final protocol, including without limitation short list of high-performing trialists/sites, suggested optimal site activation curve, and/or CRO insights and QA CRO site lists. Regarding to ongoing clinical trials, the present invention can be used to monitor competition, track site performance, forecast enrolment rates and cycle time and recommend operational adjustments.

### EXAMPLES

The present invention will be better understood by reference to the examples as follows, but those skilled in the art will readily appreciate that the specific examples detailed are only for illustrative purpose, and are not meant to limit the present invention as described herein, which is defined by the claims following thereafter.

It is noted that the transitional term “comprising”, which is synonymous with “including”, “containing” or “characterized by”, is inclusive or open-ended, and does not exclude additional, un-recited elements or method steps.

### Example 1

A clinical trial protocol for a rare respiratory disease included, among others, the following patient inclusion criteria:
1. The patent can be either male or female. The age shall be 18 to 75 years old at the time of signing the informed consent.
2. The patient has a body mass index (BMI) of 18.0 to 35.0 kg/m².
3. The patient has a historical diagnosis of asthma, as per the Global Initiative for Asthma (GINA) 2018 update.
4. The patient has a confirmed historical diagnosis of ABPA, as per the Modified International Society for Human and Animal Mycology (ISHAM) working group 2013 criteria.
5. The patient is currently considered to be in one of the following stages of ABPA:
   a) Stage 2 (Response), b) Stage 4 (Remission), c) Stage 5a (Treatment-dependent ABPA), or d) Stage 5b (Glucocorticoid-dependent asthma).
6. The patient has a serum immunoglobulin (Ig) E≥1000 IU/mL during screening (Visit 1 or Visit 2).

In addition, the clinical trial protocol also included, among others, the following patient exclusion criteria:
1. The patient has current tobacco or inhaled marijuana use or history of smoking tobacco or marijuana within the last 6 months before screening.

Our analysis of selected data is summarized in Table 1. As shown, the synthesized baseline patient characteristics suggested that the age of the patient can be up to 80. It means that the protocol with a maximum age of 75 unnecessarily rejected some eligible patients for such clinical trial, and the patient enrollment efficiency may be affected. With an amendment adjusting the maximum eligible age, the patient enrollment efficiency can be improved.

TABLE 1
Synthesized Baseline Patient Characteristics

| | | “Synthesized” Baseline | | | | | | Data from Real Trials | |
|---|---|---|---|---|---|---|---|---|---|
| | | Patient Characteristics | | | | | | for “Synthesis” | |
| | | | | | | | Percentage | Derived | Derived |
| | | | | | | Number of | of | from | from |
| | | | | | | patients | Patients | #patients | number of |
| Category | Variable | Mean | SD | Minimum | Maximum | (N) | (%) | data | #trial arms |
| | Age (yrs), mean | 37.2 | 12.7 | | | | | 222 | 6 |
| | (SD) | | | | | | | | |
| | Age (yrs), range | | | 21 | 80 | | | 645 | 9 |
| | Age at onset (y), | | | 2 | 68 | | | 417 | 4 |
| | range | | | | | | | | |
| | male | | | | | 35 | 54% | 406 | 13 |
| | Weight kg | | | 45.1 | 97.3 | | | 228 | 6 |
| | BMI, mean, | 27 | | 17 | 37 | | | 59 | 2 |
| | range | | | | | | | | |
| | Duration of | | | 0.3 | 25 | | | 245 | 7 |
| | asthma (yrs), | | | | | | | | |
| | range | | | | | | | | |
| Diagnosis | Aspergillus skin | | | | | 59 | 93% | 244 | 6 |
| method | test n (5) | | | | | | | | |
| Diagnosis | Positive | | | | | 35 | 55% | 244 | 6 |
| method | Aspergillus | | | | | | | | |
| | precipitins, | | | | | | | | |
| | n (%) | | | | | | | | |
| Severity | Normal (FEV1 > | | | | | 17 | 27% | 174 | 5 |
| | 80%) n (%) | | | | | | | | |
| Severity | Mild obstruction | | | | | 20 | 32% | 266 | 7 |
| | (FEV1 60%- | | | | | | | | |
| | 80%) n (%) | | | | | | | | |
| Severity | Moderate | | | | | 13 | 21% | 253 | 6 |
| | obstruction | | | | | | | | |
| | (FEV1 40%- | | | | | | | | |
| | 60%) n (%) | | | | | | | | |
| Severity | Severe | | | | | 12 | 19% | 253 | 6 |
| | obstruction | | | | | | | | |
| | (FEV1 < 40%) | | | | | | | | |
| | n (%) | | | | | | | | |
| Spirometry | FEV1 (%), | 66.9 | 23.2 | | | | | 77 | 3 |
| | mean (SD) | | | | | | | | |
| Spirometry | FEV1 (%), | | | 47 | 136 | | | 126 | 5 |
| | range | | | | | | | | |
| Spirometry | FEV1 (L), mean | 1.76 | 0.82 | | | | | 271 | 8 |
| | (DS) | | | | | | | | |
| Spirometry | FEV1 (L), range | | | 1 | 3.2 | | | 115 | 2 |
| Spirometry | FVC, (L), mean | 2.76 | 0.90 | | | | | 216 | 6 |
| | (SD) | | | | | | | | |
| Spirometry | FVC (%), range | | | 61.4 | 95.9 | | | 113 | 4 |
| Spirometry | FEV1/FVC, | 67.5 | 13.40 | | | | | 161 | 4 |
| | mean (SD) | | | | | | | | |
| Spirometry | FEV1/FVC (%) | | | 41 | 85 | | | 228 | 6 |
| | range | | | | | | | | |
| Symptom | Brownish-black | | | | | 14 | 22% | 253 | 6 |
| | mucus plugs | | | | | | | | |
| Symptom | Hemoptysis | | | | | 16 | 25% | 253 | 6 |
| Symptom | High-attenuation | | | | | 22 | 34% | 274 | 8 |
| | mucus | | | | | | | | |
| | Smoking, | | | | | 39 | 61% | 59 | 2 |
| | Never, n (%) | | | | | | | | |
| | Smoking, Ever, | | | | | 25 | 39% | 59 | 2 |
| | n (%) | | | | | | | | |
| Comorbid | Bronchiectasis | | | | | 47 | 74% | 388 | 12 |
| Condition | | | | | | | | | |
| Immunological | A fumigatus- | 31.9 | 22.8 | | | | | 149 | 3 |
| | specific IgE | | | | | | | | |
| | levels, kUA/L, | | | | | | | | |
| | mean, SD | | | | | | | | |
| Immunological | A. fumigtus | | | 0.9 | 87.5 | | | 245 | 7 |
| | specific IgE | | | | | | | | |
| | levels, kUA/L, | | | | | | | | |
| | range | | | | | | | | |
| Immunological | Total IgE levels | | | 346 | 35435 | | | 279 | 10 |
| | IU-mL-1 | | | | | | | | |
| Immunological | Eosinophil blood | 12.3 | 9.31 | | | | | 131 | 2 |
| | count (/mm3), | | | | | | | | |
| | mean (SD) | | | | | | | | |
| Immunological | Total eosinophil | | | 60 | 2221 | | | 245 | 7 |
| | count, cells/\hat{ }L, | | | | | | | | |
| | range | | | | | | | | |
| Medication | Inhaled | | | | | 64 | 100% | 266 | 7 |
| | corticosteroids | | | | | | | | |
| Medication | leukotriene | | | | | 26 | 40% | 253 | 6 |
| | receptor | | | | | | | | |
| | antagonists | | | | | | | | |
| Medication | long acting beta | | | | | 64 | 100% | 266 | 7 |
| | agonist | | | | | | | | |

As shown, the synthesized baseline patient characteristics suggested that the BMI of patient can be up to 37 kg/m². It means that the protocol with a maximum BMI value of 35 kg/m² unnecessarily rejected some eligible patients for such clinical trial, and the patient enrollment efficiency may be affected. With an amendment adjusting the maximum BMI value, the patient enrollment efficiency can be improved. Indeed, obesity is common for such respiratory disease.

The synthesized baseline patient characteristics also suggested the minimum total IgE level can be as low as 346 IU/mL. It indicates that the protocol with a minimum total IgE level of age of 1000 IU/mL unnecessarily rejected some eligible patients for such clinical trial and limited to a much smaller target population. With an amendment adjusting the minimum total IgE level, the patient enrollment efficiency can be improved and the target population can be significantly expanded.

The synthesized baseline patient characteristics also revealed that 39% of patients in the selected data had smoking history. It indicates that the protocol excluding smoking patient unnecessarily rejected some eligible patients for such clinical trial. It is recommended to amend the protocol by eliminating the exclusion criteria.

### Example 2

In this example, an analysis is conducted on one actual clinical trial which had gone through a few protocol amendments. Table 2-1 shows the initial protocol dated Oct. 23, 2005. Tables 2-2, 2-3 and 2-4 show the protocols amended on May 5, 2006, Jun. 14, 2006 and Aug. 6, 2006, respectively.

TABLE 2-1
Clinical trial protocol on Oct. 23, 2005

| | |
|---|---|
| Inclusion | Age 18 to 45 years old |
| Criteria | Diagnosis of MS within the past 3 years according to the |
| | McDonald criteria (2001) |
| | Baseline EDSS score between 0 and 3.5 inclusively |
| Exclusion | Unable to produce T cell vaccine |
| Criteria | Disease-modifying treatment for MS during the past 60 days |
| | Diagnosis of progressive-relapsing, secondary progressive |
| | or primary progressive MS |
| | Planned pregnancy |
| | Any prior treatment with total lymphoid irradiation, |
| | cladribine, T cell or T cell receptor vaccination |

TABLE 2-2
Clinical trial protocol on May 5, 2006

| | |
|---|---|
| Inclusion | Age 18 to 50 years old |
| Criteria | Diagnosis of “High Risk” CIS defined in protocol or |
| | Diagnosis of MS within the past 5 years according to the |
| | McDonald criteria (2005) |
| | Baseline EDSS score between 0 and 3.5 inclusively |
| Exclusion | Unable to produce T cell vaccine |
| Criteria | Disease-modifying treatment for MS during the last 30 days |
| | and 60 days for steroidal treatment |
| | Diagnosis of progressive-relapsing, secondary progressive |
| | or primary progressive MS |
| | Planned pregnancy |
| | Any prior treatment with total lymphoid irradiation, |
| | cladribine, T cell or T cell receptor vaccination |

TABLE 2-3
Clinical trial protocol on Jun. 14, 2006

| | |
|---|---|
| Inclusion | Age 18 to 50 years old |
| Criteria | Diagnosis of “High Risk” CIS defined in protocol or |
| | Diagnosis of MS within the past 5 years according to |
| | the McDonald criteria (2005) |
| | Baseline EDSS score between 0 and 3.5 inclusively |
| Exclusion | Unable to produce T cell vaccine |
| Criteria | Disease-modifying treatment for MS during the last 30 days |
| | and 60 days for steroidal treatment |
| | Diagnosis of progressive-relapsing, secondary progressive |
| | or primary progressive MS |
| | Planned pregnancy, currently pregnant or breastfeeding |
| | Any prior treatment with total lymphoid irradiation, |
| | cladribine, T cell or T cell receptor vaccination |

TABLE 2-4
Clinical trial protocol on Aug. 6, 2006

| | |
|---|---|
| Inclusion | Age 18 to 55 years old |
| Criteria | Presence of myelin reaction T cells at screening |
| | Diagnosis of CIS with screening MRI that fulfills the |
| | Barkhof criteria-dissemination in space or Diagnosis |
| | of MS within the past 10 years according to the McDonald |
| | criteria (2005) |
| | Baseline EDSS score between 0 and 5.5 inclusively |
| Exclusion | Unable to produce T cell vaccine |
| Criteria | Disease-modifying treatment for MS during the last 30 days |
| | and 60 days for steroidal treatment |
| | Diagnosis of progressive-relapsing, secondary progressive |
| | or primary progressive MS |
| | Planned pregnancy, currently pregnant or breastfeeding |
| | Any prior treatment with total lymphoid irradiation, |
| | cladribine, T cell or T cell receptor vaccination |

According to Tables 2-1 to 2-4, the age was kept as a parameter for inclusion criteria. The minimum age was kept as the same without any further adjustment, i.e., the value was set as 18. The value for maximum age was initially set as 45, subsequently changed to 50 for the May 5, 2006 and the Jun. 14, 2006 protocols, and finally changed to 55 for the Aug. 6, 2006 protocol. Similarly, the value for the EDSS score was originally set as 0-3.5, while it was amended to 0-5.5 at the protocol dated Aug. 6, 2006. Our analysis indicated that these three amendments were moving the parameter values to the modal values. If one amendment was necessary and conducted to include all necessary adjustments, it can eliminate another 2 amendments which would reduce the cost and accelerate the progress of the clinical trial. If the original clinical trial protocol was better designed at the very beginning, no clinical trial amendments would be necessary.

What is claimed is:

1. A method for evaluating and amending a protocol of a target clinical trial associated with a disease wherein said protocol has a plurality of parameters each having a value, comprising:
1) obtaining a set of inclusion and exclusion criteria of a target clinical trial associated with a disease;
2) transforming said criteria to a filter for filtering a master database storing patient data in historical clinical trials associated with said disease, generating a subdatabase storing filtered data;
3) constructing a set of baseline characteristics based on said filtered data stored in said subdatabase;
4) conducting a quantitative analysis by comparing said protocol to said set of baseline characteristics; and
5) making a recommendation based on said quantitative analysis; wherein said recommendation comprises one of the following:
a) no protocol amendment is needed if said quantitative analysis indicates that said protocol would achieve one or more objectives of said target clinical trial; and
b) a protocol amendment is needed if said quantitative analysis indicates one of the following:
(1) at least one parameter of said protocol shall be removed or added; and
(2) at least one parameter value of said protocol shall be adjusted,
wherein said protocol amendment would help said target clinical trial achieve one or more objectives according to said quantitative analysis.

2. The method of claim 1, wherein said set of baseline characteristics comprises all parameters for patient screening in said historical clinical trials.

3. The method of claim 1, wherein said set of baseline characteristics comprises all key parameters with commonly used values for patient screening in said historical clinical trials.

4. The method of claim 3, wherein said commonly used values comprise modal values.

5. The method of claim 1, wherein said quantitative analysis quantifies one or more effects of said target clinical trial caused by an adjustment assuming said adjustment is made.

6. The method of claim 5, wherein said one or more effects are or associated with one or more of the following:
a) patient enrollment time,
b) number of patients,
c) number of investigator sites,
d) efficiency of enrolling patients at either site or clinical trial level,
e) cost, and
f) a confidence level to achieve any one of a)-e).

7. The method of claim 6, wherein said efficiency of enrolling patients at site level is characterized by Gross Site Enrollment Rate (GSER), Adjusted Site Enrollment Rate (ASER), or Site Effectiveness Index (SEI), wherein GSER is defined as number of patients enrolled per site per unit of time, ASER is defined as:
$$A S E R = \frac{T E}{{\int }_{i = 1}^{N} ( E {t}_{i} - S {t}_{i} )}$$
and SEI is defined as:
$$SEI = \frac{{\int }_{i = 1}^{N} ( {Et}_{i} - {St}_{i} )}{( {Et}_{s} - {St}_{s} ) \times {N}_{max}} ,$$
wherein TE is Total Enrollment, Etᵢ is the time or date the ith site was closed for patient enrollment, and Stᵢ is the time or date the ith site was opened for patient enrollment Nₘₐₓ is the maximum number of investigator sites opened for enrollment during the patient enrollment, Etₛ is the time or date the historical clinical trial was closed for patient enrollment, Stₛ is the time or date the historical clinical trial was opened for patient enrollment.

8. The method of claim 6, wherein said efficiency of enrolling patients at clinical trial level is characterized by Clinical Trial Enrollment Rate (CTER), wherein CTER is defined as number of patients enrolled per unit of time.

9. The method of claim 1, wherein said set of baseline characteristics is provided in a table format.

10. The method of claim 1, wherein said set of baseline characteristics is constructed on a graphical user interface, which allows a user to, in view of importance and priority, add or remove one or more parameters therein.

11. The method of claim 1, wherein said set of baseline characteristics is constructed on a graphical user interface virtually displaying one or more quantitative analysis results upon selection by a user on said graphical user interface, wherein said one or more quantitative analysis results are from said quantitative analysis.

12. The method of claim 11, wherein said one or more quantitative results displayed on said graphical user interface comprise a bar chart, a bubble chart or both.

13. The method of claim 11, wherein said graphical user interface displays a bar chart and a bubble chart, wherein said bar chart profiles values of a selected parameter and frequencies of said values in historical trials, and wherein said bubble chart comprises a plurality of bubbles each representing one clinical trial, and size of bubble reflects enrollment cycle time, wherein when said user selects a value from said values for said selected parameter in said bar chart, the bubble chart highlights all clinical trials with said selected value for said selected parameter.

14. The method of claim 1, wherein said master database further stores other data from other sources comprising conference abstracts, oral presentations, publications, and regulatory filings.

15. The method of claim 14, wherein said other data from said other sources is tabulated and stored in a uniform format for data filtering.

16. The method of claim 1, wherein said one or more objectives comprise one or more of the following:
a) enrollment is completed within a target time;
b) a target number of patients is enrolled,
c) a target number of investigator sites is registered,
d) a target efficiency of enrolling patients at either site or clinical trial level is achieved,
e) a target budget is contained, and
f) a target confidence level to achieve any one of a)-e) is satisfied.

17. The method of claim 1, wherein, when a protocol amendment is needed, the clinical trial protocol is amended by adjusting a parameter value to modal value in said set of baseline characteristics.

18. The method of claim 1, wherein, when a protocol amendment is needed, said protocol is amended to achieve a new objective.

19. The method of claim 18, wherein said new objective is set in view of importance or priority of said target clinical trial from time to time.

20. The method of claim 1, wherein when a protocol amendment is needed, said protocol is amended in a way that minimum incidences of amendments would be needed.
